(19) United States
(12) Patent Application Publication
(10) Pub. No.: US 2020/0222613 A1
(43) Pub. Date: Jul. 16, 2020

(54) Control Apparatus For Blood Treatment Apparatus, And Blood Treatment Apparatus

(71) Applicant: Fresenius Medical Care Deutschland GmbH, Bad Homburg (DE)

(72) Inventors: Reiner SPICKERMANN, Wasserlosen-Burghausen (DE); Carsten MUELLER, Euerbach (DE)

(21) Appl. No.: 16/623,945

(22) PCT Filed: Jun. 15, 2018
(86) PCT No.: PCT/EP2018/066026

§ 371 Date: Dec. 18, 2019

(30) Foreign Application Priority Data
Jun. 19, 2017 (DE) 10 2017 113 393.3

(51) Int. Cl. A61M 1/28; A61M 1/36

(52) U.S. Cl. CPC A61M 1/282; A61M 1/3607; A61M 2205/587; A61M 2205/502; A61M 2205/18

(57) ABSTRACT

The present disclosure relates to a control apparatus, configured to control or regulate a blood treatment apparatus when the latter is connected in signal communication with the control apparatus for a blood treatment that already has been performed or is to be performed. The control apparatus includes at least a switch with a switching section and a signal processing unit. The signal processing unit is configured to receive signals from the foot switch and configured to emit signals to components of the blood treatment apparatus to control or regulate the blood treatment apparatus.

### CROSS-REFERENCE TO RELATED APPLICATIONS

The present application is the national stage entry of International Patent Application No. PCT/EP2018/066026, filed on Jun. 15, 2018, and claims priority to Application No. DE 10 2017 113 393.3, filed in the Federal Republic of Germany on Jun. 19, 2017, the disclosures of which are expressly incorporated herein in its entirety by reference thereto.

### TECHNICAL FIELD

The present disclosure relates to a control apparatus, a blood treatment apparatus, and a method.

### BACKGROUND

Control apparatuses for blood treatment apparatuses, the control apparatuses being operated by a switch or a touch screen, are known from practice.

### SUMMARY

The present disclosure relates to a control apparatus which is configured to control or regulate a blood treatment apparatus when this blood treatment apparatus is connected in signal communication for a blood treatment that has already been performed or is to be performed, with the control apparatus.

The control apparatus comprises at least a foot switch with at least a switching section and/or is connected therewith in signal communication and/or is correspondingly configured for a signal transmission between control apparatus and foot switch.

Further, the control apparatus comprises at least a signal processing unit which is configured to receive signals from the foot switch. It is also designed to emit signals to components of the blood treatment apparatus to control or regulate the latter.

The present disclosure in addition relates to a blood treatment apparatus which comprises at least a control apparatus.

A method described herein is used to end or change an emitted alarm signal, in particular an alarm tone, issued by a blood treatment apparatus which apparatus is assigned to a second patient and/or to a further patient, and/or said method is used to confirm a notification issued by said blood treatment apparatus. The method is executed by a medical professional wearing a first pair of gloves that he/she has put on to treat a first patient who differs from the second patient.

The method further encompasses confirming, ending or changing an alarm signal, in particular an alarm tone, issued by the blood treatment apparatus which apparatus is assigned to the second patient, and/or said method encompasses confirming a notification issued by said blood treatment apparatus.

This action is performed—preferably exclusively—by the foot switch of the blood treatment apparatus which apparatus is assigned to the second patient. The medical professional (in general: the user) keeps the first pair of gloves on.

In all the following descriptions, the use of the term “may be” or “may have” is to be understood as synonym for “preferably is” or “preferably has” etc. and is intended to explain embodiments.

Embodiments described herein may comprise one or several of the features named above or below. The features named herein may be subject of embodiments in arbitrary combinations, if the person skilled in the art does not recognize a specific combination as being technically impossible.

Embodiments are also subject of the dependent claims and their embodiments.

Whenever there is mention of numerical phrases herein, the person skilled in the art understands them as specification of a numerical lower limit. As long as there is no underlying objection recognized by the person skilled in the art, the latter thus for example always reads “at least a(n)” or “at least one” when there is mention of “a(n)” or “one”, too. This understanding is also comprised by the present invention as well as the interpretation that a numerical phrase like, for example, “one” is alternatively meant to be “exactly one”, wherever this is recognized as being technically possible by the person skilled in the art. Both are encompassed by the present invention and apply to all numerical phrases used herein.

Whenever there is mention of an embodiment herein, it is an exemplary embodiment.

The specifications “top” and “bottom” are herein to be understood as absolute or relative space details, when or if the person skilled in the art is in doubt, the details relating to the orientation of the respective component during its common use. “Bottom” may be closer to the center of the earth than “top”.

In some embodiments, the control apparatus comprises a switching section which is a touch switch to be actuated by a user or it comprises such a touch switch.

In some embodiments, the touch switch comprises at least two switch positions or at least two switching areas which emit two switch signals to the signal processing unit, the switch signals being different from each other.

In some embodiments, the switching section of the control apparatus is a touchless, in particular an optical or inductive switch to be actuated by a user in a touchless way, or it comprises such switch.

In some embodiments, the switching section or sensor and/or the foot switch are arranged, e.g., beneath the blood treatment apparatus, a bottom section of a housing or the like, or beneath a patient's bed, a lying surface or the like (for example, at the foot of the bed).

In some embodiments, the switching section or sensor and/or the foot switch are arranged to be actuated from below or from (or at) an underside, or to be actuated by gestures, movements, touches or the like, taking place from below the switching section, sensor or foot switch.

In some embodiments, the touchless switching section and/or the signal processing unit are designed to translate gestures into signals to control or regulate the blood treatment apparatus.

In some embodiments, the signal processing unit is configured to determine an actuation of the switching section or to transmit a corresponding signal only if or when this switching section is being or has been actuated for a predetermined minimum period or according to a predetermined actuation pattern. Such an actuation pattern may for example be an actuation taking place repeatedly in succession, e.g., twice within a predetermined time span or, for example, after the foot of the user has been detected in the proximity of the switching section for a predetermined period of time.

In some embodiments, the control apparatus comprises a foot switch which has at least a lighting device, for example an LED.

In some embodiments, the lighting device or the lighting devices is or are designed to emit light in more than just one color of light.

In some embodiments, the lighting device is configured to emit multicolored light.

In some embodiments, the control apparatus additionally comprises a hand switch. This hand switch may be provided, for example, as a controller or touch screen switch to be actuated by the hand or a different body part, for example, the elbow.

In some embodiments, the signal processing unit is designed to emit a signal when the switching section is actuated and—where appropriate—in addition when the hand switch is actuated, the signal effecting at least an action of a respective treatment apparatus which is connected with the control apparatus.

Such actions include, for example, switching on or halting or stopping an action, as well as starting, ending or continuing a blood treatment, such as a dialysis treatment. Further, such an action may include ending a triggered alarm or an alarm message, just like increasing or decreasing a pumping rate, in particular a feed rate or the speed (e.g. rotational speed) of a blood pump. Apart from that, the signal processing unit may request a confirmation the user should make. For example, the change between treatment modes and treatment tempos, such as for example slow start, priming, rinsing or reinfusion may be counted among the effected actions.

In some embodiments, such actions for example do not—or not exclusively—include switching or stopping a fluid pump, for example in connection with confirming or stopping an alarm, actuating clamps, operating further elements or devices and/or starting, ending or continuing a blood treatment, such as a dialysis treatment.

In some embodiments, the signal processing unit is not designed to effect or act on a control of a fluid circuit or fluid flow upon an actuation of the switching section, in particular not to act on an element which is in connection with a fluid circuit or a fluid line or which acts on a fluid line.

In some embodiments, the signal processing unit is designed to prompt one or only exactly one of the aforementioned activities, upon an actuation of the switching section, such as acting on an alarm, e.g. by confirming or switching off the alarm and/or by differently reacting to the alarm.

In some embodiments, the signal processing unit is designed to react to or act on—optionally only—an alarm state upon an actuation of the switching section such as in order to end an alarm state, to confirm and/or to react differently to the alarm or to the alarm device triggering the alarm (said alarm device being an example of a component of the blood treatment apparatus for controlling or closed-loop controlling it) and/or to its sub-devices (as lamps, speakers etc.).

In some embodiments, the signal processing unit is designed to react to or act on upon actuation of the switching section solely to the alarm device (as an example of a component of the blood treatment apparatus for controlling or closed-loop controlling it) and/or on its sub-devices.

In several embodiments, an alarm is to be understood as a notification for the medical professional, in others not.

In some embodiments, the control apparatus further includes a display which may show parameters for treatment setting and/or requests upon the user and is optionally programmed for this purpose.

In some embodiments, the signal processing unit is configured to emit signals to the display so that requests upon the user to actuate the foot switch are displayed.

In certain embodiments, the signal processing unit is designed to emit signals to the foot switch to light up a lighting device which is assigned to a specific switching section. These light signals may in particular serve as request upon the user to actuate the associated switching section.

In some embodiments, the control apparatus has a transmission device which transmits signals between the foot switch and the signal processing unit in a wireless way.

In some embodiments, the blood treatment apparatus comprises a stand, wherein the at least one foot switch is integrated in the stand.

In some embodiments, the control apparatus is a closed-loop control apparatus.

In some embodiments, the control apparatus is the control apparatus or closed-loop control apparatus of the blood treatment apparatus, in particular its only control apparatus or closed-loop control apparatus or main control apparatus or closed-loop control apparatus.

In some embodiments, the blood treatment apparatus is an apparatus for therapeutic apheresis, for liver support therapy, for oxygenation, for dialysis (hemofiltration, hemodiafiltration, hemodialysis) or for peritoneal dialysis.

In several embodiments, the method encompasses providing and/or assigning a blood treatment apparatus to the second patient.

Some or all of the embodiments may comprise one, several or all of the advantages mentioned before and/or hereafter.

Acting on an alarm device by the control device as described herein may be advantageous as follows: Often enough, the medical professional is busy taking care of a first patient, while an alarm starts or is issued at the treatment apparatus adjacent to the first patient by which apparatus a second patient is being treated. “Adjacent” is to be understood here broadly: the second patient does not necessarily have to be directly adjacent; said patient could also be for example five beds further or even be in another room. The professional who has taken note of the alarm or possibly of its cause, relevance, etc., is in practice endeavoring to first and as quickly as possible end the alarm state, such as to end the disturbing alarm tone or to prevent that colleagues, who likewise became aware of the alarm, unnecessarily make their way to the second patient. However, as the medical professional who realized the alarm is still caring for the first patient whereby he/she is wearing gloves as requested for reasons of hygienic, said professional must in practice first remove this first pair of gloves (and discard them as they are disposables) and must put on the next, i.e. second pair of gloves for ending the alarm state or the alarm tone prior to being allowed to act on the treatment apparatus of the second patient to end the alarm. Likewise, before the medical professional can return to the first patient to continue his/her treatment interrupted because of the alarm, he/she must remove (and discard) the second pair of gloves, which meanwhile have been put on in order to avoid possible contamination so that to put on a new, i.e. sterile, third pair of gloves. This procedure must be repeated each time for each patient, even with only a short or brief operation of the treatment apparatus, such as confirming an alarm. In this way, due to hygiene reasons, a considerable amount of hygiene articles such as gloves may be consumed for the simple confirmation of a notification or an alarm. The latter, however, is not the case if e.g. alarms could be confirmed not by hand rather by foot as the present invention proposes this in several embodiments. Thus, it is possible for the professional caring for the first patient to keep his/her first pair of gloves on and to end an alarm on the treatment apparatus of the second or further patient by a foot switch and therefore without using his/her hands, without having to touch a touch screen, a hand switch or the like of this treatment apparatus with the hands. Creating—especially infectious—garbage can thereby be prevented advantageously. In addition, the time and effort of the professional and his/her colleagues can be saved.

It may be of advantage that the user, such as the dialysis nurse or the doctor, may operate the blood treatment apparatus or some functions hereof at least also by the foot switch. At least for switching operations that may be carried out by the foot it is not necessary that the user actuates a switch by his/her hand. Thus, manual switching operations are eliminated, in which thus the switch or switching areas have to be touched with the hand, which in turn may advantageously help lower the risk of germ transmission.

As the number of manual switching operations the user has to carry out may be decreased, since by use of the foot switch described herein switching operations may be “transferred to the foot”, also the number of changes of gloves necessary for operating the blood treatment apparatus, in particular while the user looks after the patient, may be decreased. Taking off gloves to actuate, e.g., the touch screen and the subsequent putting on of new gloves or changing of gloves may thus may be reserved to those few moments, in which such glove procedure cannot be avoided, even if the foot switch is used. In this way, considerable amounts of disposable gloves may be saved for example during dialysis, which apart from saving time also helps to save costs for purchase, storage and disposal.

Eventually, the user may take care of the patient with both hands, for example when placing needles and at the same time controlling the blood treatment apparatus—by the foot switch.

### BRIEF DESCRIPTION OF THE FIGURES

The present invention is exemplarily explained with regard to the accompanying drawing in which identical reference numerals refer to the same or similar components. The following applies in the drawing:

FIG. 1 shows an exemplary blood treatment apparatus with a control apparatus with a foot switch in an exemplary embodiment, in a schematically simplified form.

### DETAILED DESCRIPTION OF THE FIGURES

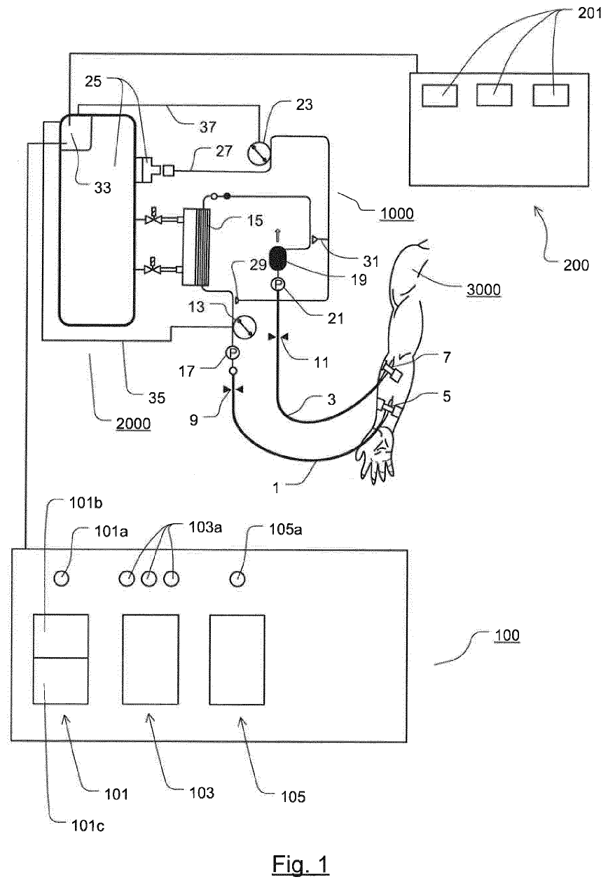

FIG. 1 shows a blood treatment apparatus 2000 which purely exemplarily is a dialysis device. For the same reason, the components of the blood treatment apparatus 2000 named hereafter are each to be understood as being optional.

In FIG. 1, an extracorporeal blood circuit 1000, connected with a blood treatment apparatus 2000, with a double needle access to the vascular system of a patient 3000 is shown. The extracorporeal blood circuit 1000 has an arterial line section 1 and a venous line section 3.

The arterial line section 1 is connected with the vascular system of the patient 3000 via an arterial patient access, such as for example shown in FIG. 1, by an arterial connection needle 5. The venous line section 3 is connected with the vascular system of the patient 3000 via a venous patient access, such as for example shown in FIG. 1, by a venous connection needle 7.

The arterial line section 1 has an arterial patient hose clamp 9, the venous line section 3 has a venous patient hose clamp 11, or it is each connected herewith, respectively.

The extracorporeal blood circuit 1000 is inserted in a blood pump 13 of the blood treatment apparatus 2000. The blood pump 13 is exemplarily designed as a roller pump. The blood pump 13 delivers blood of the patient 3000 through the extracorporeal blood circuit 1000.

The extracorporeal blood circuit 1000 has a blood treatment device 15, for example a hemodialyzer, blood filter, or the like.

As shown in FIG. 1, an arterial pressure sensor 17 is located at the arterial line section 1 upstream (relative to the feed direction common during a blood treatment) the blood pump 13.

A venous drip chamber 19 and a venous pressure sensor 21 are present or arranged in the venous line section 3 downstream the venous drip chamber 19.

By means of a second optional feed device, for example a substituate pump 23, a substituate liquid can be introduced into the extracorporeal blood circuit 1000 from a substituate liquid supply 25 via a substituate supply line 27 at an addition point 29 for pre-dilution for substituate liquid and/or at an addition point 31 for post-dilution for substituate liquid. The substituate pump 23 is exemplarily designed as a roller pump here.

The blood treatment apparatus 2000 has a signal processing unit 33. The signal processing unit 33 is connected with the blood pump 13 via a control line 35 and it is connected with the substituate pump 23 via a control line 37.

The signal processing unit 33 may for example control a feed rate or a feed volume of the blood pump 13 depending on operating parameters of the substituate pump 23, e.g., its feed rate or feed volume.

The control lines 35 and 37 may permit a bidirectional communication between the signal processing unit 33 and the blood pump 13, or the substituate pump 23, respectively.

FIG. 1 shows a connection of the blood treatment apparatus 2000 with the vascular system of the patient 3000. Such a connection is not part of the blood treatment apparatus 2000, but only represents its special design and preferred use.

FIG. 1 also shows a first embodiment of the control apparatus which encompasses in addition to the signal processing unit 33 at least one foot switch 100.

The foot switch 100 is in signal connection with the signal processing unit 33 by a wire (as exemplarily shown in FIG. 1) or wirelessly. The foot switch 100 may be a part of the blood treatment apparatus 2000 and, for example, be integrated into its housing, which is not shown in FIG. 1, and which optionally has rollers for its onward movement.

The foot switch 100 may be integral with the blood treatment apparatus 2000. As an alternative, it may be provided separately and, e.g., be easily accessible for the user caring for the patient and be pushed out of the way under the edge of the patient's bed or the patient table.

The foot switch 100 may have one or several switching sections. Exemplarily, in FIG. 1 three switching sections 101, 103 105 are shown. Each of the switching sections 101, 103 105 may thereby optionally have one or several lighting devices 101a, 103a, 105a, which are explained in more detail hereafter.

The switching sections 101, 103 are optionally designed as touch switches and thus have to be touched in order to inform the signal processing unit 33 that they have been actuated. Optionally, they have to be pressed under application of force. For this purpose, they may be designed, e.g., as mechanically actuated switches or be part of those, those mechanically actuated switches being similarly pressable like a common light switch. They may be designed as touch switch, similar to a touch screen switch for manual input.

The switching section 101 is thereby designed as a double switch with two switching areas 101b, 101c.

The switching areas 101b, 101c and/or the signal processing unit 33 may be configured, for example in the form of a rocker or as part of a toggle, to increase, a parametric value of the blood pump 13 or a different device of the blood treatment apparatus 2000 when actuating one of the switching areas, for example 101b, and to decrease the same when actuating the other switching area, in this example 101c (or vice versa). Thus, the switching section 101 may be used as an up/down switch.

The switching section 103 has—contrary to the switching section 101—only one switching area and may be used, e.g., as an on/off switch. As an alternative, it may be used as mere off switch, which, when being actuated, can or does turn off, an alarm sound, an alarm signal or an alarm state, but optionally not turn it on.

The switching section 105 is a touchless switching section, different from the switching sections 101. 103. The switching section 103 does not have to be touched to be actuated, but it may be touched as an option. Being a touchless switching section, it can be designed as optical switch, as light barrier, as gesture controlled switch which reacts to foot movements like tapping the foot tip on the ground, lifting the foot tip twice, or the like, as switch which evaluates a proximity, such as the proximity of a foot, etc.

The switching section may be designed and/or arranged to be actuated from below, or from the underside of the switching section, in an optical, mechanical, or other way. For example, the switching section may be mounted beneath the blood treatment apparatus or beneath the patient's bed. It may be actuated by a foot movement taking place underneath the switching section/sensor.

Each switching section 101, 103, 105 may be designed and/or configured to give the user feedback that an actuation has taken place. This may, be a tactile, optical, and/or acoustic feedback confirming the user that an actuation has taken place. The feedback may be carried out by the lighting device 101a, 103a, 105a and/or through a corresponding representation or information on a display 200, see below.

The assignment of the switching sections 101, 103, 105 with functions by a corresponding configuration of the signal processing unit 33 is not limited to the examples given above.

The lighting devices 101a, 103a, 105a may optionally be designed as LED or any other device emitting visible light. They may be each of the same type or may also be differently designed lighting devices.

Each of the lighting devices 101a, 103a, 105a may be or comprise a multicolored lighting device, for example an LED. Alternatively, each may comprise several lighting devices, as for example LEDs, which may each emit different colors of light.

As can further be taken from FIG. 1, the blood treatment apparatus 2000 may optionally have at least a display 200 or be in signal connection with it by a cable, or in a wireless way.

The display 200 may have at least one hand switch 201 to be actuated with the hand or a different body part, for example the elbow, for example a touch screen switch or a touch screen controller. As an alternative, or in addition, the blood treatment apparatus 2000 may have a hand switch 201 at a location other than the display 200.

The hand switch 201 may be configured to pass on entries that have been made by hand, in the form of suitable signals, to the signal processing unit 33. In doing so, the reaction of the signal processing unit 33 may optionally be the same as if the signal came from the foot switch 100. This way, the control apparatus may allow to enter certain entries either by hand or elbow or by foot.

Optionally, the control apparatus may be configured to display orders on the display 200 which request the user to actuate the foot switch 100 or a specific switching section 101, 103, 105, in particular to actuate it in a special way. For this purpose, verbal comments may be displayed on the display 200, as an alternative or in addition, illustrations of the foot switch 100 and/or of the switching section 101, 103, 105 to be actuated may be shown. The user can thus be guided through processes by the display 200. If he/she is thereby requested to execute instructions or issue confirmations, he/she can do so by the foot switch 100, without having to touch the display 200 or other components of the blood treatment apparatus 2000. In this way, the use of disposable gloves can be diminished. Furthermore, some interruptions of the treatment process which can be attributed to taking off contaminated gloves to actuate a switch of the blood treatment apparatus 2000 to continue the treatment of the patient and the subsequent pulling on of gloves may be prevented.

Optionally, the signal processing unit 33 may be configured to emit signals to the foot switch 100.

For example, the signal processing unit 33 may light up one or several of the lighting devices 101a, 103a, 105a by such signals, as requests upon the user to please actuate the corresponding switching section 101, 103, 105.

Thus, the user can recognize on the basis of a lit lighting device 101a, 103a or 105a that a certain switching section 101, 103, 105 is to be actuated, for example, to confirm the continuation of the treatment.

In doing so, the user may be requested on the basis of the number of lit lighting devices 101a, 103a or 105a, their assignments to certain switching sections 101, 103, 105, the period of time in which they are each lit, the color in which they are lit, the lighting mode in which they emit light (flashing, permanently, intermittently, intermittently with varying gaps, e.g., speeding up, etc.), to actuate one or several of the switching sections 101, 103, 105 assigned to the lighting devices 101a, 103a, 105a.

The switching sections 101, 103, 105 or any other switching section mentioned herein may optionally be redundant switching sections. Herein, under these are to count switching sections of the foot switch 100, the function of which—being effected when being actuated by the signal processing unit 33—can also be effected by alternatively actuating a switching section not being part of the foot switch 100 but for example of the display 200. For example, the switching section 101 may be configured to effect an acceleration of the blood pump 13 when it is being actuated by the signal processing unit 33. At the same time, a touch screen switch, like the hand switch 201, may be provided which is also configured to effect an acceleration of the blood pump 13 upon actuation. “Redundant” switching sections are thus those which are not configured exclusively to effect one and the same function. Such a redundancy may advantageously allow to actuate or operate the blood treatment apparatus 2000 conveniently and from a central point e.g. by a display 200 or a control bar. In addition, the user can switch to the foot switch 100 for certain functions. This can facilitate the work, for example, in situations in which he or she needs both hands to care for the patient and/or in which he or she would have to take off or change gloves specifically in order to actuate the manual switching section, e.g., during handling of the catheter, puncturing, etc.

### REFERENCE NUMERAL LIST

1 arterial line section
3 venous line section
5 arterial connection needle
7 venous connection needle
9 arterial patient hose clamp
11 venous patient hose clamp
13 blood pump
15 blood treatment device
17 arterial pressure sensor
19 venous drip chamber
21 venous pressure sensor
23 substituate pump
25 substituate liquid supply
27 substituate liquid supply line
29 addition point for pre-dilution
31 addition point for post-dilution
33 signal processing unit
35 control line to the blood pump
37 control line to the substituate pump
100 foot switch
101 switching section
101a lighting device
101b switching area
101c switching area
103 switching section
103a lighting device
105 switching section
105a lighting device
200 display
201 hand switch
1000 extracorporeal blood circuit
2000 blood treatment apparatus
3000 patient

1-19. (canceled)

20. A control apparatus, configured to control or regulate a blood treatment apparatus when in signal connection with the blood treatment apparatus, the control apparatus comprising:
a foot switch having at least one switching section; and
a signal processing unit configured to receive signals from the foot switch and configured to emit signals to components of the blood treatment apparatus to control or regulate the blood treatment apparatus.

21. The control apparatus according to claim 20, wherein the switching section is or comprises a touch switch, wherein the touch switch is configured to be actuated by a user.

22. The control apparatus according to claim 21, wherein the touch switch has at least two switch positions or at least two switching areas for emitting different switching signals to the signal processing unit.

23. The control apparatus according to claim 20, wherein the switching section is or comprises a touchless switch.

24. The control apparatus according to claim 23, wherein the touchless switch is an optical or inductive switch.

25. The control apparatus according to claim 23, wherein the touchless switching section and/or the signal processing unit are configured to translate gestures into signals; and wherein the processing unit is configured to control or regulate the blood treatment apparatus based on the signals.

26. The control apparatus according to claim 20, wherein the signal processing unit is configured to:
(i) detect an actuation of the switching section;
(ii) transmit an according signal when actuated for a predetermined minimum duration; or
(iii) transmit an according signal according to a predetermined actuation pattern.

27. The control apparatus according to claim 20, wherein the foot switch comprises a lighting device.

28. The control apparatus according to claim 27, wherein the lighting device is configured to emit light in more than just one color.

29. The control apparatus according to claim 27, wherein the lighting device is configured to emit multicolored light.

30. The control apparatus according to claim 20, further comprising a hand switch configured to be actuated by a body part of a user.

31. The control apparatus according to claim 20, wherein the signal processing unit is configured to emit a signal when the switching section is actuated or when the switching section and a hand switch are actuated, the signal being emitted to effect at least one action of a blood treatment apparatus connected with the control apparatus, wherein the at least one action is from the group encompassing:
turning on, halting, or stopping an action;
starting, ending, or continuing a dialysis treatment;
terminating a triggered alarm or an alarm signal;
increasing or decreasing a pump rate, a feed rate, or speed of a blood pump;
emitting a confirmation by a user, the confirmation being requested by the signal processing unit; and
switching between treatment modes or treatment tempos.

32. The control apparatus according to claim 20, further comprising a display to display settings of treatment parameters and/or of requests upon the user.

33. The control apparatus according to claim 32, wherein the signal processing unit is configured to emit signals to the display to display a request to the user to actuate the foot switch.

34. The control apparatus according to claim 20, wherein the signal processing unit is configured to emit signals to the foot switch, to activate a lighting device assigned to a specific switching section.

35. The control apparatus according to claim 34, wherein the activation of the lighting device notifies the user to actuate the assigned switching section.

36. The control apparatus according to claim 20, with a transmission device wirelessly transmits signals between the foot switch and the signal processing unit.

37. A system comprising:
a blood treatment apparatus; and
a control apparatus configured to control or regulate the blood treatment apparatus when in signal connection with the blood treatment apparatus, the control apparatus comprising:
a foot switch having at least one switching section; and
a signal processing unit configured to receive signals from the foot switch and configured to emit signals to components of the blood treatment apparatus to control or regulate the blood treatment apparatus.

38. The system according to claim 37, wherein the blood treatment apparatus further comprises a stand, wherein the foot switch is integrated in the stand.

39. A method for ending or changing an alarm signal issued by a second blood treatment apparatus assigned to a second patient, the method comprising:
wearing a first pair of gloves which have been put on for treatment of a first patient; wherein a first blood treatment apparatus is assigned to the first patient; and
confirming, ending or changing an alarm signal issued by the second blood treatment apparatus by a foot switch electronically connected to the second blood treatment apparatus, wherein the first pair of gloves is kept on.
